(19) United States
(12) Patent Application Publication
(10) Pub. No.: US 2023/0075697 A1
(43) Pub. Date: Mar. 9, 2023

(54) COMPOSITION COMPRISING AN EXTRACT OF ROSE BUSH SUCKER

(71) Applicant: L'OREAL, Paris (FR)

(72) Inventors: Mathieu CHABRILLANGEAS, Chevilly La Rue (FR); Thierry COTTON, Gigors-Et-Lozeron (FR); Sandrine MATHON-DUBOST, Chevilly La Rue (FR)

(21) Appl. No.: 17/784,253

(22) PCT Filed: Dec. 18, 2020
(86) PCT No.: PCT/EP2020/087214

§ 371 Date: Jun. 10, 2022

(30) Foreign Application Priority Data
Dec. 20, 2019 (FR) FR1915249

(51) Int. Cl. A61K 8/9789; A61K 8/34; A61Q 19/08

(52) U.S. Cl. CPC A61K 8/9789; A61K 8/345; A61Q 19/08; A61K 2800/262; A61K 2800/522

(57) ABSTRACT

The present invention relates to a composition, preferably cosmetic, comprising:
at least one rosewood extract comprising polyphenols, and
at least one solvent selected from:
   polyols comprising at least three hydroxy functions,
   mixtures of at least one polyol comprising at least three hydroxy functions and water, and
   mixtures of at least one polyol comprising at least three hydroxy functions and at least one diol
   comprising from 2 to 4 carbon atoms,
   the weight ratio of rosewood extract to solvent being strictly less than 7:13.

The present invention relates to a composition, preferably cosmetic, comprising an extract of rosewood rich in polyphenols.

Human skin is formed of two main layers which are the dermis and the epidermis which covers the surface of the dermis. The natural human epidermis is primarily composed of three types of cells which are keratinocytes, which are largely predominant, melanocytes, and Langerhans cells. Each of these cell types contributes via the specific functions thereof to the key role played by the skin in the body, particularly the role of protecting the body from external attacks (climate, ultraviolet rays, tobacco, etc.), known as the “barrier function”.

The epidermis is a keratinized stratified pavement epithelium formed of 90% keratinocytes. The progressive differentiation of the cells of the basement membrane, which separates the dermis from the epidermis, to the surface of the epidermis particularly comprises the differentiation of keratinocytes which migrate to the skin surface where they desquamate.

Aging of the epidermis primarily manifests through the reduction of the thickness thereof. Epidermal atrophy is the result of the slowdown of keratinocyte proliferation and the accumulation of senescent keratinocytes. The stratum corneum becomes dull.

Desquamation is a natural phenomenon associated with the fact that the epidermis, which forms the upper layer of the kin, is constantly regenerating. The epidermis is formed of several cell layers, the deepest whereof is the basal layer formed of undifferentiated cells. Over time, these cells will differentiate and migrate to the surface of the epidermis forming the various layers thereof, until corneocytes, which are dead cells eliminated by desquamation, are formed on the surface of the epidermis. This loss on the surface is offset by the migration of cells from the basal layer to the surface of the epidermis. This is the perpetual renewal of the skin. Forced removal of the stratum corneum accelerates renewal and helps combat aging.

At the same time, these cells continue the differentiation thereof, the final stage whereof is the corneocyte. In fact, these are dead cells which form the final layer of the epidermis, i.e., the outermost layer also known as the stratum corneum.

Moreover, in the course of chronological and/or actinic aging, the skin is subjected to oxidative stress, which can be summarized as the oxidative attack of all cellular biological macromolecules (DNA, lipids, proteins) by Reactive Oxygen Species (ROS): superoxide radical O₂, hydrogen peroxide H202. hydroxyl radical HO°, singlet oxygen ¹02 or nitrogen (RNS: peroxynitrite ONQO, nitrogen monoxide (NO). This oxidative stress, which can be physiological or caused by external agents such as UV, pollution or tobacco, induces cell and tissue damage, which contributes to skin aging.

For the skin, UVA rays (320-400 nm) are the main source of oxidative skin stress.

They are absorbed by chromophores which transmit the energy required by oxygen to bring it to the reactive singlet state (¹02). UVA rays also induce the reduction of O₂ to the superoxide anion (O₂”), in turn rapidly dismutated into hydrogen peroxide (H₂O₂) by the superoxide dismutase (SOD) enzyme. H₂O₂ is then reduced to the hydroxyl radical (OH°) in the presence of ferrous ion (Fenton reaction) or by the superoxide radical anion (Haber-1s Weiss). The hydroxyl radical is the most reactive and can remove a hydrogen from any neighboring molecule RH, to give the alkyl radical (R°), the precursor form of the peroxyl and alkoxyl radicals (ROO° and RO° ), which are involved in the propagation phase during self-oxidation. These reactive forms of oxygen are interdependent.

It is therefore clearly understood that it is important to combat oxidative skin stress to limit skin aging.

Antioxidant active agents are proposed to combat oxidative skin stress.

The latter particularly include polyphenols. These compounds are particularly flavonoid polyphenols such as epigallocatechin, epicatechin 3-gallate, epigallocatechin 3-gallate, baicalin, taxifolin, quercetin, hesperetin or genistein; or non-flavonoid polyphenols, such as resveratrol. Of these polyphenols, some are derived from plant extracts.

However, the plant extracts comprising a large quantity of polyphenols generally exhibit solubility problems in water. Furthermore, they give the composition obtained a turbid appearance.

The inventors have now discovered that combining a specific plant extract, comprising a large quantity of polyphenols, with a specific glycol such as glycerol, makes it possible to solubilize these polyphenols in a hydrophilic medium, and obtain a non-turbid and stable composition.

Thus, the present invention relates to a composition comprising:

at least one rosewood extract comprising polyphenols, and

at least one solvent selected from:
polyols comprising at least three hydroxy functions, wherein said polyol(s) is then the only solvent of the composition,
mixtures of at least one polyol comprising at least three hydroxy functions and water, and
mixtures of at least one polyol comprising at least three hydroxy functions and at least one diol comprising from 2 to 4 carbon atoms,
the weight ratio of rosewood extract to solvent being strictly less than 7:13.

The composition according to the invention is preferably cosmetic. Indeed, as shown in the examples, a polyol comprising at least three hydroxy functions such as glycerol makes it possible to solubilize the polyphenols contained in the plant extract, in order to obtain a composition according to the invention which is homogeneous, while being transparent and stable.

The term “stable” composition denotes a composition which exhibits no settling after centrifugation, in particular at least up to 24h after centrifugation for 1h at 900g at ambient temperature. Furthermore, a composition is considered to be “stable” if it exhibits no settling after centrifugation up to two months at ambient temperature, at 4° C., at 37° C. and 45° C.

The present invention also relates to a kit comprising:
at least one composition according to the invention; and
at least one cosmetic composition comprising at least one aqueous phase and/or at least one oily phase.

The present invention also relates to a cosmetic care method of keratin materials, preferably the skin, comprising application of a composition according to the invention on said keratin materials.

The present invention also relates to cosmetic use of a composition according to the invention to combat oxidative skin stress. The present invention also relates to cosmetic use of a composition according to the invention to combat the signs of skin aging, such as for example wrinkles or lines.

### Rosewood Extract Comprising Polyphenols

The composition according to the invention comprises at least one rosewood extract comprising polyphenols.

Polyphenols represent a family of organic molecules found extensively in the plant kingdom. The term “polyphenol” denotes a compound comprising at least two phenol groups; these phenol groups can be associated in more or less complex structures, and they are generally of high molecular weight.

Among polyphenols, mention can particularly be made of flavonoid polyphenols such as epigallocatechin, epicatechin 3-gallate, epigallocatechin 3-gallate, baicalin, taxifolin, quercetin, hesperetin or genistein; or non-flavonoid polyphenols, such as resveratrol.

The rosewood extract according to the invention is an extract rich in polyphenols. The rosewood extract comprises at least 20% by weight, preferentially at least 30% by weight of polyphenols with respect to the total weight of the extract.

Typically, the rosewood extract is obtained from rose bush stalks and/or leaves and/or suckers. Preferably, the rose used is of the genus Rosa, preferably the variety is Rosa centifolia (Rosa xcentifolia). Preferably, the rose used was grown in the South of France, and the stalks and/or leaves and/or suckers were cut at the end of the winter.

The extract is typically obtained according to the following method:
a) extracting the rose bush stalks and/or leaves and/or suckers in a hydroalcoholic mixture, preferably at a temperature less than or equal to 90° C., in order to obtain a hydroalcoholic mixture comprising the extract;
b) removing the plant debris and/or solid particles, from the hydroalcoholic mixture comprising the extract obtained in a), so as to obtain a liquid comprising the rosewood extract. Step b) can particularly comprise a primary separation step, a centrifugation step and/or a filtration step. Preferably, step b) comprises removing the plant debris, particularly by separation. from the hydroalcoholic mixture comprising the extract obtained in a), then the solid particles, particularly by centrifugation and/or filtration;
c) then, preferably, atomizing the liquid comprising the rosewood extract obtained in b), to obtain a rosewood extract comprising polyphenols in powder form.

Preferably, the rosewood extract comprising polyphenols according to the invention is presented in powder form. Typically, the powder is dark, brown to black in color.

Preferably, the rosewood extract comprising polyphenols according to the invention is present in a quantity less than or equal to 35% by weight with respect to the total weight of the composition, preferably less than or equal to 30% by weight.

Preferably, the rosewood extract comprising polyphenols according to the invention is present in a quantity of at least 0.01% by weight with respect to the total weight of the composition, preferably at least 0.05%, preferably at least 0.1%, preferably at least 1%, preferably at least 1.5% by weight, preferably at least 2% by weight.

Solvent

The composition according to the invention comprises at least one solvent selected from:
polyols comprising at least three hydroxy functions, wherein said polyol(s) is then the only solvent of the composition,
mixtures of at least one polyol comprising at least three hydroxy functions and water, and
mixtures of at least one polyol comprising at least three hydroxy functions and at least one diol comprising from 2 to 4 carbon atoms.

Furthermore, the composition according to the invention preferably comprises at least 0.01%, at least 2%, at least 4%, at least 6%, at least 10%, at least 20%, at least 35% by weight, with respect to the total weight of the composition, of polyol comprising at least three hydroxy functions.

The term polyol comprising at least 3 hydroxy functions according to the invention denotes a hydrocarbon chain having at least 2 carbon atoms, preferably from 2 to 50 carbon atoms, preferably from 3 to 20 carbon atoms, preferably having from 2 to 10 carbon atoms, and preferentially from 2 to 6 carbon atoms, and carrying at least three hydroxy groups. The polyols used in the present invention can have a mean molecular mass of less than or equal to 1000 g/mol, preferably between 85 and 500 g/mol.

The polyol comprising at least 3 functions can be a natural or synthetic polyol. The polyol can have a linear, branched or cyclic molecular structure.

The polyol comprising at least 3 hydroxy functions can be selected from glycerin and derivatives thereof, and glycols comprising at least 3 hydroxy functions and derivatives thereof. The polyol comprising at least 3 hydroxy functions can be selected from the group consisting of glycerin (or glycerol), diglycerin (or diglycerol), sugars such as sorbitol, and mixtures thereof.

More specifically, the polyol comprising at least 3 hydroxy functions is glycerin.

According to the invention, the weight ratio of rosewood extract to solvent is strictly less than 7:13 (i.e. 0.538). Preferably. the weight ratio of rosewood extract to solvent is less than or equal to 0.5, preferably less than or equal to 0.45. Preferably, the weight ratio of rosewood extract to solvent is at least 0.01, preferably at least 0.02.

When the rosewood extract according to the invention is present in a quantity of at least 1% by weight with respect to the total weight of the composition, the polyol comprising at least 3 hydroxy functions according to the invention is present in a quantity of at least 35% by weight with respect to the total weight of the composition, preferably at least 40% by weight, preferably at least 45% by weight.

When the rosewood extract according to the invention is present in a quantity of at least 1% by weight with respect to the total weight of the composition, the polyol comprising at least 3 hydroxy functions according to the invention can be present at a concentration ranging from 35% to 99% by weight, with respect to the total weight of the composition, preferably ranging from 40% to 95% by weight, and preferentially ranging from 50% to 90% by weight.

The polyol comprising at least three hydroxy functions can be used alone; it is then the only solvent. In this case, when the rosewood extract according to the invention is present in a quantity of at least 1% by weight with respect to the total weight of the composition, the polyol comprising at least 3 hydroxy functions according to the invention can be present at a concentration ranging from 35% to 99% by weight, with respect to the total weight of the composition, preferably ranging from 50% to 98.5% by weight, and preferentially ranging from 60% to 98% by weight. Especially, when the polyol comprising at least three hydroxy functions is used alone (as a single/only solvent), then the composition is devoid of any other solvent, including water and/or diols comprising from 2 as to 4 carbon atoms. Typically, when the polyol comprising at least three hydroxy functions is used alone (as a single solvent), then the composition is devoid of water, dipropylene glycol and/or pentylene glycol.

According to a further embodiment, the solvent is a mixture of at least one polyol comprising at least three hydroxy functions and water. In this case, when the rosewood extract according to the invention is present in a quantity of at least 1% by weight with respect to the total weight of the composition, the polyol comprising at least 3 hydroxy functions according to the invention can be present at a concentration ranging from 35% to 90% by weight, with respect to the total weight of the composition, and water is present at a concentration ranging preferably from 10% to 65% by weight, and preferentially ranging from 20% to 55% by weight.

According to a further embodiment, the solvent is a mixture of at least one polyol comprising at least three hydroxy functions and at least one diol comprising from 2 to 4 carbon atoms. Preferably, the diol comprising from 2 to 4 carbon atoms is a diol comprising a linear carbon chain, preferably the diol is a propanediol, preferably propane-1,2-diol.

When the rosewood extract according to the invention is present in a quantity of at least 1% by weight with respect to the total weight of the composition, the polyol comprising at least 3 hydroxy functions according to the invention can be present at a concentration ranging from 35% to 90% by weight, with respect to the total weight of the composition, preferably from 45% to 55% by weight, and the diol comprising from 2 to 4 carbon atoms is present at a concentration ranging preferably from 40% to 70% by weight, and preferentially ranging from 45% to 55% by weight.

Preferably, the composition according to the invention consists of:
at least one rosewood extract comprising polyphenols, and at least one solvent selected from:
polyols comprising at least three hydroxy functions, wherein said polyol(s) is then the only solvent of the composition,
mixtures of at least one polyol comprising at least three hydroxy functions and water, and
mixtures of at least one polyol comprising at least three hydroxy functions and at least one diol comprising from 2 to 4 carbon atoms,

the weight ratio of rosewood extract to solvent being strictly less than 7:13.

Preferably, this weight ratio of rosewood extract to solvent is less than or equal to 0.5, preferably less than or equal to 0.45. Preferably, the weight ratio of rosewood extract to solvent is at least 0.01, preferably at least 0.02.

Kit According to the Invention

The present invention also relates to a kit comprising:
at least one composition according to the invention (also referred to as “composition A”); and
   at least one cosmetic composition comprising at least one aqueous phase and/or at least one oily phase (also referred to as “composition B”).

The composition according to the invention is as described above. Preferably, this composition is packaged in a pipette, which can be broken before mixing and application on keratin materials, preferably the skin. 1 5

Typically, the composition according to the invention (composition A) is mixed with the cosmetic composition comprising at least one aqueous phase and/or at least one oily phase (composition B), then applied on keratin materials, particularly the skin.

The cosmetic composition comprising at least one aqueous phase and/or at least one oily phase can be an emulsion or a gel. Preferably, such a composition comprises an aqueous phase and an oily phase, said aqueous and oily phases being as defined above.

Preferably, such a composition is an oil-in-water emulsion,

Aqueous Phase

Composition B can comprise a physiologically acceptable aqueous medium. “Physiologically acceptable” means a medium compatible with keratin materials.

Composition B preferably comprises an aqueous medium comprising water and optionally an organic solvent soluble in water, at 25° C., selected for example from linear or branched C2-C4 alkanols such as ethanol and isopropanol, propanol, butanol; polyols; and mixtures thereof,

The polyols can be selected from the compounds having a hydrocarbon chain having as at least 2 carbon atoms, preferably from 2 to 50 carbon atoms, preferably from 4 to 20 carbon atoms, preferably from 2 to 10 carbon atoms, and preferentially having from 2 to 6 carbon atoms, and carrying at least two hydroxy groups. The polyol can be a natural or synthetic polyol. The polyol can have a linear, branched or cyclic molecular structure,

This polyol can be selected from glycerin and derivatives thereof, and glycols and derivatives thereof. The polyol can be selected from the group composed of glycerin, diglycerin, polyglycerin, diethylene glycol. propylene glycol, dipropylene glycol, butylene glycol, pentylene glycol, hexylene glycol, 1,3-propanediol, 1,5-pentanediol, octane 1,2-diol, polyethyleneglycols, particularly having from 5 to 50 ethylene oxide groups, and sugars such as sorbitol, and mixtures thereof.

Said polyol(s) can be present in a quantity ranging from 2% to 30% by weight, with respect to the total weight of composition B, preferably ranging from 3% to 25% by weight, and preferentially ranging from 5% to 20% by weight.

Composition B generally comprises from 10 to 95% by weight of water with respect to the total weight of the composition, preferably from 40 to 80%,

The quantity of organic solvent(s) can range for example from 0 to 30% by weight, preferably from 0.5 to 25% by weight, more preferably from 5 to 20% by weight, even more preferably from 10 to 22% by weight relative to the total weight of composition B.

Oily Phase

Composition B can comprise at least one oily phase. When the composition includes an oily phase, this oily phase preferably contains at least one oil, particularly a cosmetic oil.

It can further contain other fats.

By way of oils suitable for use in the composition, mention may be made for example of:
hydrocarbon oils of animal origin, such as perhydrosqualene;
hydrocarbon oils of plant origin, such as liquid fatty acid triglycerides having from 4 to 10 carbon atoms such as heptanoic or octanoic acid triglycerides or, for example, sunflower, corn, soybean, pumpkin, grape seed, sesame, hazelnut, apricot, macadamia, arara, sunflower, castor, avocado oils, caprylic/capric acid triglycerides such as those sold by Stearineries Dubois or those sold under the trade names Miglyol “810”, “812” and “818” by Dynamit Nobel, jojoba oil, shea butter oil;
esters and synthetic esters, in particular fatty acids, such as oils having formulas R1COOR2 and R1OR2 wherein R1 is the residue of a fatty acid having from 8 to 29 carbon atoms, and R2 is a hydrocarbon chain, branched or not, containing from 3 to 30 carbon atoms, such as for example Purcellin oil, isononyl isononanoate, isopropyl myristate, ethyl-2-hexyl palmitate, octyl-2-dodecyl stearate, octyl-2-dodecyl erucate, isostearyl isostearate; hydroxylated esters such as isostearyl lactate, octylhydroxystearate, octyldodecyl hydroxystearate, diisostearyl malate, triisocetyl citrate; heptanoates, octanoates, decanoates of fatty alcohols; polyol esters, such as propylene glycol dioctanoate, neopentyl glycol dlheptanoate and diethylene glycol diisononanoate; and pentaerythritol esters such as pentaerythrityl tetraisostearate or pentaerythrityl tetraoctanoate;
linear or branched hydrocarbons, with inorganic or synthetic origin, such as volatile or non-volatile paraffin oils and derivatives thereof, hydrocarbon oils with branched chain containing 10 to 20 carbon atoms such as isohexadecane, isododecane, isoparaffins and mixtures thereof, petroleum jelly, polydecenes, hydrogenated polyisobutene such as Parleam@ oil;
fatty alcohols having 8 to 26 carbon atoms, such as cetyl alcohol, stearyl alcohol, the mixture of cetyl alcohol and stearyl alcohol (cetylstearyl alcohol), octyldodecanol, 2-butyloctanol, 2-hexyldecanol, 2-undecylpentadecanol, oleic acid or linoleic acid;
partially hydrocarbon and/or silicone fluorinated oils such as those described in the document JP-A-2-295912;
silicone oils such as polymethylsiloxanes (PDMS), optionally volatile with a linear or cyclic silicone chain, liquid or pasty at ambient temperature, particularly cyclopolydimethylsiloxanes (cyclomethicones) such as cyclohexasiloxane; polydimethylsiloxanes comprising alkyl, alkoxy or phenyl pendant or silicon chain-end groups, groups having 2 to 24 carbon atoms; phenyl silicones such as phenyltrimethicones, phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, and polymethylphenylsiloxanes; or
mixtures thereof.

The quantity of oily phase can range for example from 0.1 to 30%, and for example from 1 to 20% by weight with respect to the total weight of composition B.

Composition B can also comprise conventional cosmetic adjuvants particularly selected from gelling agents, surfactants, preservatives, electrolytes, pH adjusters, ionic or non-ionic thickening agents, moistening agents, anti-foaming agents, perfumes, active agents, particularly anti-aging agents, fillers or any ingredient usually used in the cosmetic and/or dermatological field.

The present invention also relates to a cosmetic care method of keratin materials, preferably the skin, comprising application of a composition according to the invention on said keratin materials.

The present invention also relates to cosmetic use of a composition according to the invention to combat oxidative skin stress. The present invention also relates to cosmetic use of a composition according to the invention to combat the signs of skin aging, such as for example wrinkles or lines.

We will now give concrete examples illustrating the invention, but that are in no way limitative.

In the examples, the temperature is ambient temperature (20° C.) expressed in degrees Celsius unless mentioned otherwise, and the pressure is atmospheric pressure, unless mentioned otherwise.

In the examples, quantities of the ingredients of the compositions are given as a % by weight relative to the total weight of the composition (% w/w),

### Example 1: Solubilization of a Rosewood Extract in Various Solvents

200g of rosewood extract, obtained from Rosa centifoia leaves and stalks according to the method as described in the description, and comprising from 30% to 50% by weight with respect to the total weight of the extract of polyphenols, in powder form, was mixed at ambient temperature with various solvents, with a rotor-stator, before testing various evaluation criteria.

The evaluation criteria are as follows:
solubility of the mixture after rotor-stator;
stability of the dispersion 24h after centrifugation for 1h, at 900g; and
degree of transparency of the dispersion (if relevant).

The compositions and the results are presented in Tables 1 to 3.

Mixtures A to E, G to J, M and 0 are comparative,

Mixtures F, L and N are according to the invention (marked by an asterisk).

TABLE 1

| Ingredients | | | | | |
|---|---|---|---|---|---|
| (% w/w) | Mixture A | Mixture B | Mixture C | Mixture D | Mixture E |
| Rosewood extract | 2 | 2 | 2 | 2 | 2 |
| Water | 98 | | | | |
| Ethanol | | 98 | | | |
| Propane-1,2-diol | | | 98 | | |
| Isododecane | | | | 98 | |
| Pentylene glycol | | | | | 98 |
| APPEARANCE | Turbid | Biphasic, | Biphasic, | Biphasic, | Biphasic, |
| before | liquid and | settlement | settlement | settlement | settlement |
| centrifugation | settlement | | | | |
| Centrifugation | Settlement | Settlement | Settlement | Settlement | Settlement |
| | sediment | sediment | sediment | sediment | sediment |

TABLE 2

| Ingredients | | | | | |
|---|---|---|---|---|---|
| (% w/w) | Mixture F* | Mixture G | Mixture H | Mixture I | Mixture J |
| Rosewood extract | 2 | 2 | 2 | 2 | 2 |
| Glycerol | 98 | 24.5 | | | |
| Dipropylene glycol | | 73.5 | 98 | | |
| Propane-1,2-diol | | | | | |
| Pentaerythrityl | | | | 98 | |
| tetraethylhexanoate | | | | | |
| Isononyl | | | | | 98 |
| isononanoate | | | | | |
| Solubility | NTR | OK at T0 | OK at T0 | Not OK Phase | Not OK Phase |
| | | Bubbly-turbid | Bubbly-turbid | separation from | separation from |
| | | appearance | appearance | T0 | T0 |
| Centrifugation at | NTR No | Settlement | | | |
| T24 h (1 g, 900 g) | settlement | | | | |
| Transparency | Transparent | Turbid | Turbid | | |
| Conclusion | OK | Not OK | Not OK | Not OK | Not OK |

Conclusion:

Glycerin is the only solvent, of the solvents tested, enabling complete solubilization, stable in centrifugation and resulting in a translucent mixture,

TABLE 3

| Ingredients | | | | |
|---|---|---|---|---|
| (% w/w) | Mixture L* | Mixture M | Mixture N* | Mixture O |
| Rosewood extract | 2 | 2 | 2 | 2 |
| Glycerol | 98 | 49 | 49 | 49 |
| Dipropylene glycol | | | | 49 |
| Propane-1,2-diol | | | 49 | |
| Pentylene glycol | | 49 | | |
| Solubility | NTR | OK at T0 | OK at T0 | OK at T0 |
| | | Bubbly-turbid | Bubbly-turbid | Bubbly-turbid |
| | | appearance | appearance | appearance |
| Centrifugation at | NTR No | Settlement | OK | Settlement |
| T24 h (1 g, 900 g) | settlement | | | |
| Transparency | Transparent | | Transparent | |
| Conclusion | OK | Not OK | OK | Not OK |

Conclusion:

Glycerin can be used alone, or indeed in combination with 50% propane-1,2-diol to obtain a mixture that is stable in centrifugation and translucent.

### Example 2: Determination of the limit of solubility of rosewood extract in glycerin

As shown in example 1, the use of glycerin as a solvent of the rosewood extract makes it possible to solubilize the extract at ambient temperature, and obtain a translucent mixture.

Various mixtures M1 to M7 were produced, with various quantities of extract and glycerin.

The evaluation criteria are as follows:
solubility of the mixture after rotor-stator (appearance); and
stability of the dispersion 24h after centrifugation for 1h, at 900g.

The results are presented in Table 4.

TABLE 4

| Ingredients | Mixture | Mixture | Mixture | Mixture | Mixture | Mixture | Mixture |
|---|---|---|---|---|---|---|---|
| (% w/w) | M1 | M2 | M3 | M4 | M5 | M6 | M7 |
| Rosewood | 15 | 20 | 50 | 30 | 35 | 40 | 15 |
| extract | | | | | | | |
| Glycerol | 85 | 80 | 50 | 70 | 65 | 60 | 35 |
| Water | — | — | — | — | — | — | 50 |
| APPEARANCE | Homogeneous | Homogeneous | Saturation | Homogeneous | Foamier | Inhomogeneous | Homogeneous |
| | dark | dark | dispersion | dark | but | foamy | brown |
| | brown | brown | | brown | turbid | lighter | very fluid |
| | thick | thick | | thick | brown | brown | liquid |
| | liquid | liquid | | liquid | smooth- | smooth- | |
| | | | | | flowing | flowing, | |
| | | | | | thick | almost | |
| | | | | | paste | hard | |
| | | | | | | paste | |
| Centrifugation | NTR (= | NTR | | NTR | Limit | Limit of | NTR |
| | no | | | | of | visual | |
| | settlement) | | | | visual | perception | |
| | | | | | perception | | |
| Conclusion | OK | OK | Not | OK | Not | Not OK | OK |
| | | | OK | | OK | | |

Conclusion:

The limit of solubilization of the rosewood extract is situated at about 30%.

The best solubilization results are obtained with 15% to 25% by weight of extract with respect to the total weight of the composition.

### Example 3: Composition and kit according to the invention

The composition described hereinafter in Table 5, according to the invention, can be used as a booster, in particular at 6%, in a mixture with the cosmetic composition described in Table 6 (serum). The mixture is obtained before being applied on the skin.

Typically, the composition of Table 5 (booster) can be packaged in a pipette, which is broken before mixing with the cosmetic composition of Table 6 (serum), then applied on the skin.

TABLE 5

| Ingredients (% w/w) | Composition according to the invention |
|---|---|
| Rosewood extract | 2 |
| Glycerol | 98 |

TABLE 6

| Ingredient | % (w/w) | Phase |
|---|---|---|
| MICROBIOLOGICALLY PURE DEIONIZED WATER | q.s. 100 | A |
| EDTA IN 30% AQUEOUS SOLUTION | 0.25 | A |
| OCTANE-1,2-DIOL | 0.3 | A |
| Preservative | q.s. | A |
| GLYCERIN | 3 | A |
| 1,3-BUTYLENE GLYCOL | 5 | A |
| PROPANE-1,3-DIOL | 3 | A |
| POLY ACRYLAMIDOMETHYL PROPANE SULFONIC ACID | 0.3 | B |
| PARTIALLY NEUTRALIZED WITH AMMONIA AND | | |
| HIGHLY CROSSLINKED (AMMONIUM | | |
| POLYACRYLOYLDIMETHYL TAURATE) | | |
| Xanthan gum | 0.1 | B |
| SODIUM HYALURONATE | 0.4 | B |
| PENTAERYTHRITYL TETRAOCTANOATE | 3.2 | C |

The above composition was prepared according to the following method: P_{GP}-2₆,T₁

Phase A is mixed in a stirrer for example MAXILAB TURBOTEST tank bottom rotor/stator turbine sold by OLSA at a speed of 1000 rpm, at ambient temperature until the solution is homogeneous;
Phase B is introduced into phase A in a stirrer at a speed of 2500 rpm at ambient temperature to obtain a homogeneous gel;
Phase C is incorporated into mixture A+B with the stirrer at a speed of 2500-3000 rpm to obtain good homogenization of the solution.

1. A composition comprising:
at least one rosewood extract comprising at least 20% by weight with respect to the weight of the extract, of polyphenols, and
at least one solvent selected from:
polyols comprising at least three hydroxy functions, wherein said polyol(s) is then the only solvent of the composition,
mixtures of at least one polyol comprising at least three hydroxy functions and water, and mixtures of at least one polyol comprising at least three hydroxy functions and at least one diol comprising from 2 to 4 carbon atoms,
the weight ratio of rosewood extract to solvent being strictly less than 7:13.

2. The composition according to claim 1, wherein the rosewood extract comprising polyphenols is present in a quantity less than or equal to 35% by weight with respect to the total weight of the composition.

3. The composition according to claim 1, wherein
the weight ratio of rosewood extract to solvent is less than or equal to 0.5.

4. The composition according to claim 1, wherein the rosewood extract comprises at least 30% by weight with respect to the weight of the extract, of polyphenols.

5. The composition according to claim 1, wherein the rosewood extract is obtained from rose bush stalks and/or leaves and/or suckers.

6. The composition according to claim 1, wherein the polyol comprising at least 3 hydroxy functions comprises a hydrocarbon chain having at least 2 carbon atoms.

7. The composition according to claim 1, wherein the polyol comprising at least 3 hydroxy functions is selected from glycerin and derivatives thereof, and glycols comprising at least 3 hydroxy functions and derivatives thereof.

8. The composition according to claim 1, wherein when the rosewood extract according to the invention is present in a quantity of at least 1% by weight with respect to the total weight of the composition, the polyol comprising at least 3 hydroxy functions is present in a quantity of at least 35% by weight with respect to the total weight of the composition.

9. The composition according to claim 1, wherein the diol comprising from 2 to 4 carbon atoms is a diol comprising a linear carbon chain.

10. A cosmetic care method of keratin materials comprising applying a composition according to claim 1 on said keratin materials.

11. A cosmetic method for combatting oxidative skin stress, comprising applying a composition according to claim 1 on skin.

12. A cosmetic method for combatting the signs of skin aging comprising applying a composition according to claim 1.

13. A kit comprising:
at least one composition according to claim 1; and
at least one cosmetic composition comprising at least one aqueous phase and/or at least one oily phase.

14. The composition according to claim 2, wherein the weight ratio of rosewood extract to solvent is less than or equal to 0.5.

15. The composition according to claim 1, wherein the weight ratio of rosewood extract to solvent is at least 0.01.

16. The composition according to claim 2, wherein the weight ratio of rosewood extract to solvent is at least 0.01.

17. The composition according to claim 3, wherein the weight ratio of rosewood extract to solvent is at least 0.01.

18. The composition according to claim 2, wherein the rosewood extract comprises at least 30% by weight with respect to the weight of the extract, of polyphenols.

19. The composition according to claim 3, wherein the rosewood extract comprises at least 30% by weight with respect to the weight of the extract, of polyphenols.

20. The composition according to claim 5, wherein the rose used is of the Rosa genus.
